(19) United States
(12) Patent Application Publication
(10) Pub. No.: US 2012/0114576 A1
(43) Pub. Date: May 10, 2012

(54) Method for preventing or treating yellowish discoloration of skin

(75) Inventors: Yuki Ogura, Yokohama-shi (JP); Tomohiro Kuwahara, Yokohama-shi (JP); Tetsuji Hirao, Yokohama-shi (JP)

(73) Assignee: Shiseido Company, Ltd.

(21) Appl. No.: 13/137,242

(22) Filed: Jul. 29, 2011

Related U.S. Application Data
(60) Provisional application No. 61/369,178, filed on Jul. 30, 2010.

(51) Int. Cl. A61K 8/00; A61Q 19/08

(52) U.S. Cl. 424/62

(57) ABSTRACT

This invention relates to a method for preventing or treating skin discoloration of a subject by inhibiting carbonylation of dermal proteins of the subject who is in need of prevention or treatment for skin discoloration.

### TECHNICAL FIELD

This invention relates to a method for preventing or treating skin discoloration of a subject by inhibiting carbonylation of dermal proteins of the subject who is in need of prevention or treatment for skin discoloration.

### BACKGROUND ART

Skin color is one of the major factors that determine our esthetic skin characteristics. Among various skin color determinants of human skin hemoglobin, oxygenated or reduced, and melanin constitute the major ones [1]. Melanin, which is produced by melanocytes in the epidermis, is transferred to the surrounding keratinocytes, whereas hemoglobin present in the erythrocytes circulates through the dermis and subcutis. Particularly that in the capillary loops of the papillary dermis plays an important role in determining the skin color. Moreover, other chromophores such as bilirubin and its derivatives, which are dark yellow substances derived from degradation of the heme structure, and carotenoids, such as carotene, constituting yellowish chromophores, derived from diet may occasionally contribute to the skin color, and even skin surface morphology, reflective index, and scattering substances, can influence the exterior appearance of the skin [2]. These components contributing to the skin color influence greatly the esthetic aspect of the skin. Usually aging of facial skin constitutes the most visible portion of the body, which is greatly influenced by photo-aging recognized as dark spots, wrinkles, and sagging. In addition, elderly Japanese people often exhibit a yellowish skin change in their facial skin. Such a yellowish color change has been confirmed objectively by measuring skin color with colorimetry [3-5]. Nishimori et al. suggested a relationship between yellowish skin discoloration and elastin degeneration occurring in photo-aged facial skin [5]. In fact, clinicians are familiar with unique yellowish discolored patches induced by the abnormal accumulation of unique elastic fibers in the lesions of pseudoxanthoma elasticum. However, exact substances responsible for the diffuse yellowish change noted in the aged facial skin, particularly in Asians, has not been identified yet.

It is possible to assume that quantitative and/or qualitative alteration of melanin pigment or above-mentioned yellow chromophores may cause such yellowish change in aged facial skin of the Japanese. Moreover, recently, qualitative changes of various skin constituents occurring in photo-aging have been reported such as protein modification by glycation [6, 6b] and/or carbonylation [7, 8]. It is well established in the field of food chemistry that glycated protein can be formed via Maillard reaction between protein and sugar, and some of them are associated with the formation of dark-colored pigments, although such a chemical structure of glycation adducts is highly heterogeneous [9]. In the skin, it has been shown that glycated protein exists in the dermis as well as in the epidermis, whose level is reported to be increased in patients with diabetes mellitus [10, 11].

On the other hand, carbonylation is another type of protein modification found in our body including the skin, which can be introduced by its reaction with various kinds of aldehydes. As such reactive aldehydes of exogenous origin, acrolein is well known to be derived from cigarette smoke [12]. However, reactive aldehydes can also be formed endogenously, for example, oxidative degradation of polyunsaturated fatty acids, which are ubiquitously found in our body, can lead to the formation of various aldehydes, such as acrolein [12-14] and 4-hydroxynonenal (4-HNE) [15, 16]. These aldehydes can be formed in the skin in inflammation or in the degradation of skin surface sebum [17]. Since these aldehydes are highly reactive with other tissue components, they may not be abundantly isolated, but can be detected as a stable adduct with protein. In fact, carbonyl moieties introduced by the reaction of aldehydes with protein can be detected by a chemical reaction with hydrazide derivatives. Thus, in practice, labeled hydrazide derivatives have been quite often used to detect carbonyl modification of protein in biological tissues. Age-associated accumulation of carbonyl proteins has been described in the liver and brain, and interestingly, its increased levels are also reported in senescence-accelerated diseases [18-22]. In the skin, it has been reported that carbonyl protein is detected in the stratum corneum, particularly in the upper portion [23] of the sun-exposed area [24, 25]. The carbonyl modification of the stratum corneum results in the alteration of its physicochemical properties, such as a decrease in its water holding capacity [26], optical transparency [27], and extensibility of the skin [28]. Moreover, carbonyl protein is also accumulated in the dermis of photo-aged skin [7, 8]. However, such involvement of the accumulation of carbonyl proteins in yellowish change has remained to be elucidated.

### DISCLOSURE OF THE INVENTION

### Problems to be Solved by the Invention

It has been known that facial skin discoloration is caused by aging or oxidative stress. Photo-aged facial skin is characterized by various unique features such as dark spots, wrinkles, and sagging. Moreover, elderly people, particularly Asians, are well known to show a yellowish skin color change (i.e., yellowish discoloration) with photo-aging. Since yellowish skin discoloration causes a loss of transparent skin, and it is considered a problem in skin appearance. Therefore, the problem to be solved by the present invention is to prevent or treat skin discoloration, in particular yellowish skin discoloration.

### Means for Solving the Problems

Normal skin samples excised from the face, abdomen and buttock of variously aged Japanese were separated into the epidermal and the dermal portions. These skin samples were histologically examined for carbonyl modification. Moreover, an in vitro constructed dermis model composed of a contracted collagen gel was treated with acrolein or 4-hydroxynonenal. All of these samples were also studied colorimetrically. In the result, it has been surprisingly found that only the dermal samples obtained from the photo-aged facial skin exhibited a yellowish color, whereas dermis obtained from abdomens or buttocks have not been showed such a yellowish discoloration. The upper layer of the dermis that revealed the yellowish color showed elastosis whose elastic fibers were found to colocalize with carbonyl protein as detected by a labeled hydrazide, as well as by immunohistochemically by using the antibody against acrolein adduct. Experimental induction of carbonyl modification in a dermis model in vitro by long-term treatment with acrolein or 4-hydroxynonenal was found to show the appearance of the yellowish change which was also proven by an increase in b* value of colorimetry. It was more pronounced than that induced by glycation. These results strongly suggest that carbonyl modification of the dermis is involved in the production of the yellowish color change that is noted in the photo-aged facial skin. Further, it has been surprisingly found that the carbonylation of proteins was significantly inhibited by an olive leaf extract, a hydrolyzed pea protein and a lemon extract, and these extracts actually inhibited yellowing of a dermis model.

Therefore, the present application comprises the following embodiments:

[1] A method for preventing or treating skin discoloration of a subject by inhibiting carbonylation of dermal proteins of the subject who is in need of prevention or treatment for skin discoloration.

[2] The method according to [1], wherein the skin discoloration is yellowish skin discoloration.

[3] The method according to [1], wherein the dermal proteins are present in the upper dermis.

[4] The method according to [1], wherein the skin is sun-exposed skin.

[5] The method according to [1], wherein the skin is facial skin.

[6] The method according to [1], wherein the carbonylation of the dermal proteins is caused by aging, disease or oxidative stress.

[7] The method according to [6], wherein the disease is actinic elastosis.

[8] The method according to [6], wherein the oxidative stress is due to exposure to ultraviolet radiation, internal and/or external application of oxidative chemicals or chemical attack by reactive carbonyls derived from the degradation of lipid peroxides.

[9] The method according to [1], wherein the dermal proteins are extracellular matrix proteins.

[10] The method according to [9], wherein the extracellular matrix proteins are collagen and/or elastin.

### BRIEF DESCRIPTION OF THE DRAWINGS

FIG. 1 shows sun-exposed human isolated skin tissues (face: cases No. 1 and 2), and a sun-protected area (chest: case No. 3, groin: cases No. 4 and 5, buttock: cases No. 6 and 7). Upper columns are full-thickness skin viewed from directly above. Middle and bottom columns are epidermis and dermis, respectively, separated by 2N sodium bromide. Note that the dermal counterparts in cases No. 1 and 2 are apparently yellowish in color.

FIG. 2 shows relative coefficients of absorption of horizontal dermal sections from sun-exposed and sun-protected area black triangle, a: upper layer of the dermis from sun-exposed area (0-100 μm depth); gray triangle, b: middle layer of the dermis from sun-exposed area (100-200 μm depth); white triangle, c: lower layer of the dermis from sun-exposed area (800-900 μm depth); black circle, d: upper layer of the dermis from sun-protected area (200-300 μm depth); gray circle, e: middle layer of the dermis from sun-protected area (400-500 μm depth); white circle, f: lower layer of the dermis from sun-protected area (800-900 μm depth).

FIG. 3 shows localization of carbonylated protein (a, b) and elastic fiber (c, d) in frozen human skin sections from sun-exposed (case No. 1, a, c) and sun-protected area (case No. 3, b, d).

FIG. 4 shows immunohistochemistry of sun-exposed skin (case No. 1, a), and sun-protected skin (case No. 3, b) with the antibody for protein-bound acrolein.

FIG. 5 shows age dependent change of elastic fibers and carbonylated protein accumulation in sun-exposed skin. Localization of carbonylated proetin and Weigert's Resorcin-fuchsin stain. Sections were prepared from sun-exposed area (face) of various ages (upper column) and sun-protected area (back and buttock, lower column). Scale bar: 100 μm. Each number represents the age of the donor, and scores in parentheses shows the degree of actinic elastosis.

FIG. 6 shows experimental induction of yellowish change by carbonylation and glycation in acellular dermal models. FIG. 6A: Dermal models were treated with 10 mM acrolein or 10 mM 4-hydroxy-2-nonenal for carbonylation, or with 200 mM d-ribose or 10 mM glyoxial for glycation, at 37° C. for 9 days. Yellowish change was monitored by colorimetry and expressed in b* (mean±s.d., N=3). FIG. 6B: Appearance of the dermal models at the end of 9 days treatment. FIG. 6C: Dermal models were treated with 50 μM acrolein or 4-hydroxy-2-nonenal at 37° C. for 50 days. Yellowish change was monitored by colorimetry and expressed in b* (mean±s.d., N=3). *; p<0.05, **; p<0.01, vs control by Student t-test. FIG. 6D: Appearance of the dermal models at the end of 30 days treatment.

FIG. 7 shows an effect of inhibiting yellowing of a dermis model by an olive leaf extract.

### MODES OF CARRYING OUT THE INVENTION

The dermis of the sun-exposed facial skin, especially that in Asians, tend to exhibit more yellowish color compared with unexposed areas. In the present study performed on Japanese skin, the inventors have provided direct evidence indicating that the yellowish change in the dermis is a major determinant of the yellowish appearance of the skin noted in elderly Japanese. Namely, it is not due to the epidermal changes but that carbonyl protein produced in the upper dermis is mostly responsible to the development of the yellowish skin color alteration associated with photo-aging.

It has been well documented that the production of carbonyl protein is a hallmark of oxidative stresses, e.g., as an extrinsic factor, such as that induced by ultraviolet radiation or an external application of oxidative chemicals, or as intrinsic factors, chemical attack by reactive carbonyls derived from the degradation of lipid peroxides [18-22]. Because the skin exists as an interface between the environment and our body tissues, it is always exposed to various extrinsic hazards. Especially, the stratum corneum, the superficial layer of the skin, contains a significant amount of carbonyl proteins [23-25]. Although it may be desquamated with the epidermal turnover, a continuous exposure to oxidative stimuli results in an increase in carbonyl protein in the stratum corneum, which can lead to the alteration of its physicochemical properties [26-28].

Moreover, the dermis, which lies beneath the epidermis, is also affected directly or indirectly by the extrinsic stresses, such as solar radiation, so that there occur accumulation of carbonyl proteins in the dermis associated with solar elastosis. In the present study, the inventors have found preferential localization of carbonyl protein in the uppermost part of the dermis, being consistent with those reported before [7, 8]. Thus, it strongly suggests the involvement of solar radiation in the formation of carbonyl protein as an extrinsic stimulus. Such an alteration in the upper dermis can efficiently affect the skin color change.

Collagen and elastin that constitute the insoluble matrix proteins in the dermis have a variety of intra- and inter-molecular crosslinks in their native forms. The formation of these crosslinks is initiated by the oxidation of E-amino residue of L-lysine within collagen tropoelastin polypeptides to alpha-aminoadipic-delta-semialdehyde, which is enzymatically mediated by lysyl oxidase (EC 1.4.3.13). In addition, carbonyl moieties can also be introduced non-enzymatically via glycation. Thus, the histochemical detection of aldehydes by a labeled hydrazide cannot distinguish exogenously introduced carbonyls from those endogenously generated by lysyl oxidase, needless to say the origin of aldehydes. Therefore, in the present study, the inventors employed immunohistochemical detection of aldehyde adducts by using anti-acrolein antibody. As a result, it was clearly shown that the upper dermis of the sun-exposed area exhibited a positive staining with acrolein adducts, strongly suggesting that these acroleins are derived from the degradation of lipid peroxides that are exogenously introduced into matrix proteins in the upper dermis.

The accumulation of carbonyl protein in the dermis can be explained by the following mechanisms, i.e., the increased formation by oxidative stresses as discussed above, and decreased degradation of such protein due to their slower turnover rate in the aged skin. Since the dermal proteins generally show a slower turnover rate as compared with that of the epidermis or that of other tissues [31-33], carbonyl proteins tend to be accumulated there. In the dermal proteins, the major constituents of the extracellular matrix, such as collagen and elastin, can be a target of carbonyl modification. Therefore, it is speculated that their abundant existence and slower turnover are attributable to the preferential occurrence of carbonyl protein in the dermis associated with solar elastosis.

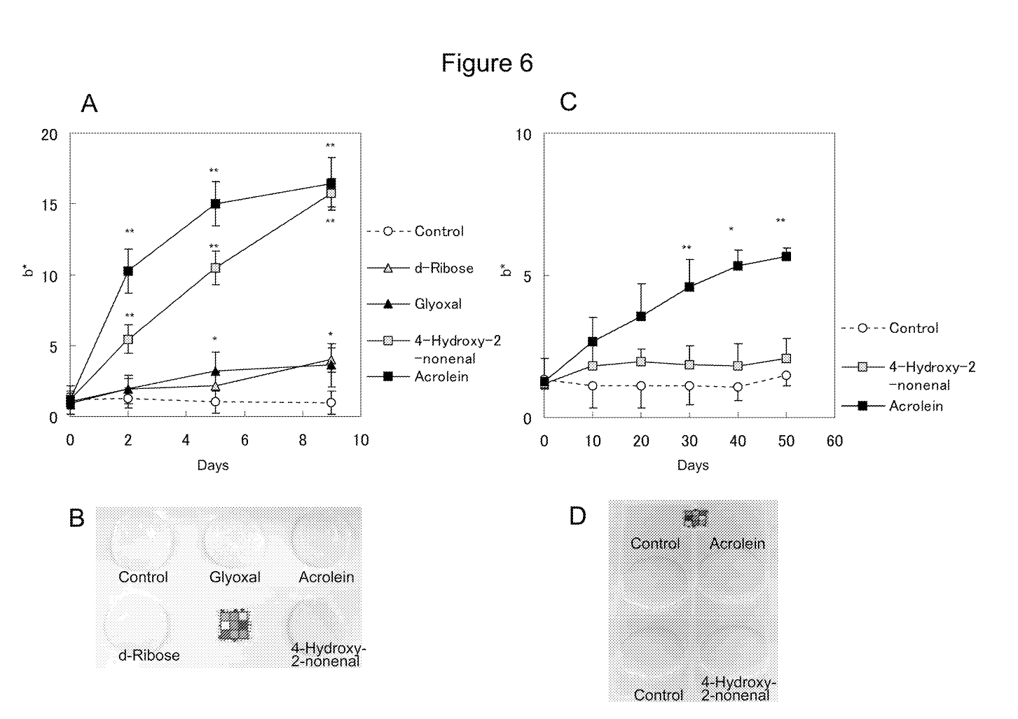

To confirm such a speculation, in the present study, the inventors conducted an in vitro study by using a contracted collagen gel as a dermis model, because of the convenience and reliability. As shown in FIG. 6, the inventors found that carbonyl modification was induced by acrolein and 4-HNE, which resulted in a significant yellowish color change compared with glycation. Furthermore, the long term treatment of a collagen gel with a lower concentration of acrolein and 4-HNE, corresponding to their physiological concentrations [12, 16], led to a slight but significant yellowish change (FIGS. 6 C and D). These findings clearly suggest that carbonyl modification of the dermis can become a major cause of the yellowish change of the dermis of photo-aged skin. The reason why a b* value is markedly increased with long-standing acrolein treatment, but not with 4-HNE, remains to be elucidated, although underlying biochemical mechanisms of each aldehyde might be slightly different.

Glycation is a similar but distinct chemical modification of protein. It has been previously reported that glycated proteins also accumulated in solar elastosis [6]. In addition, the glycated protein level in the skin is increased in diabetic patients [9-11, 34]. Since glycated protein is accumulated with aging, exhibiting a brownish color, such glycation may also be involved in the yellowish color change in the photo-aged skin. Recently, Ohshima et al. [35] reported that an age-related yellowish change in skin color was associated with melanin index and glycation. However, at Present, there is no evidence that the skin color of diabetic patients show a prominent yellowish alteration. Thus, the carbonyl modification, rather than glycation, can be one of the major causes of the yellowish change of the skin. Therefore, the present invention provides a method for preventing or treating skin discoloration of a subject by inhibiting carbonylation of dermal proteins of the subject who is in need of prevention or treatment for skin discoloration. In addition, it has been known that the carbonylation of biological proteins is associated with a wide variety of conditions or diseases. These conditions or diseases include aging, functional decline of the internal organs and brain, progeria, actinic elastosis, psoriasis, atopic dermatitis, wrinkles, freckles, skin sagging and the like. Accordingly, the present invention provides a method for preventing or treating these conditions or diseases associated with the carbonylation of biological proteins.

Further, the inventors have surprisingly found that the carbonylation of proteins was significantly inhibited by olive leaf extract, hydrolyzed pea protein and lemon extract, and these extracts actually inhibited yellowing of a dermis model.

### Olive Leaf Extract

Olive (scientific name: Olea europaea Linne) is an evergreen tree which belongs to the genus Olea of the family Magnoliaceae, is now distributed over the world in region that have a relatively high ambient temperature, and therefore is cultivated around Shodo island in Japan. Olive oil obtained by squeezing ripe fruit is widely used for edible, medicinal and cosmetic applications, and olive leaves are known to have antiseptic, antipyretic and antihypertensive actions and recently used as tea.

There is no particular limitation on the method for extracting an active ingredient from olive leaves, and an extraction method using a solvent is preferred. When the extraction is carried out, the olive leaves may be used, while the extraction of the active ingredient can be carried out at a high extraction efficiency in a short time under a mild condition when the olive leaves are provided for the extraction after grinding and shredding into powder.

There is no particular limitation on the extraction temperature, and the extraction temperature may be appropriately set depending on the size of the ground olive leaves, the type of the solvent and the like. Normally, the temperature is in a range from room temperature to z boiling point of the solvent. Also, there is no particular limitation on the extraction time, and the extraction time may be appropriately set depending on the size of the ground olive leaves, the type of the solvent, the extraction temperature and the like. Furthermore, the extraction may be carried out by stirring, being left to stand or by applying ultrasonication.

For example, the olive leaf extract can be obtained by immersing olive leaves in a solvent and extracting at room temperature or a temperature of 80° C. to 100° C. After filtering the extraction solution obtained by the extraction treatment, the resulting solution can be used as it is, or after optionally concentrating or drying, as a protein saccharification inhibitor. In this extraction treatment, the olive leaves may be used after shredding or grinding. Alternatively, green olive leaves or dried olive leaves may be used, or roasted olive leaves may be used for the extraction treatment. There is no particular limitation on the roasting method, and includes a method of roasting at 80° C. to 120° C. for 0.5 to 2 hours.

There is no particular limitation on the type of the solvent used for extraction, and the solvent is preferably water (including hot water, etc.), alcohols (for example, methanol, ethanol, 1-propanol, 2-propanol, 1-butanol and 2-butanol), glycols (for example, 1,3-butylene glycol and propylene glycol), glycerin, ketones (for example, acetone and methyl ethyl ketone), ethers (for example, diethyl ether, dioxane, tetrahydrofuran and propyl ether), acetonitrile, esters (for example, ethyl acetate and butyl acetate), aliphatic hydrocarbons (for example, hexane, heptane and liquid paraffin), aromatic hydrocarbons (for example, toluene and xylene), halogenated hydrocarbons (for example, chloroform), or mixed solvents of two or more of these solvents.

It is also possible to adopt an extraction method using a supercritical fluid as the method of extracting an olive leaf extract. There is no particular limitation on the kind of supercritical fluids, and examples thereof include nitrogen dioxide, ammonia, ethane, propane, ethylene, methanol, ethanol and the like. Moreover, an olive leaf extract is commercially available from Maruzen Pharmaceuticals Co., Ltd., and the product may be used.

### Lemon Extract

Lemon (scientific name: Citrus limon Burmann fil (Rutaceae)) is an evergreen shrub which belongs to the genus Citrus of the family Rutaceae. The lemon extract used in the protein saccharification inhibitor according to the present invention is most preferably an extract of lemon fruit (flesh and/or pericarp). Since the active ingredient is also contained in lemon leaves, stems, branches, flowers, barks, roots and the like, it is also possible to use an extract from one or more of these materials.

For example, the lemon fruit contains vitamin C, citric acid and the like in a large amount, and there is no particular limitation on the method of extracting the active ingredient from the lemon. However, an extraction method using a solvent is preferred. When the extraction is carried out, the lemon may be used as it is, while the extraction of the active ingredient can be carried out at a high extraction efficiency in a short time under a mild condition when the lemon is provided for the extraction after grinding into granules or powder.

There is no particular limitation on the extraction temperature, and the extraction temperature may be appropriately set depending on the particle size of the ground lemon, the type of the solvent and the like. Usually, the temperature is set in a range from room temperature to a boiling point of the solvents. Also, there is no particular limitation on extraction time, and the extraction time may be appropriately set depending on the particle size of the ground lemon, the kind of the solvent, the extraction temperature and the like. Furthermore, the extraction may be carried out by stirring, being left to stand, or by applying ultrasonication.

There is no particular limitation on the type of the solvent used for extraction, and the solvent is preferably water (including hot water, etc.), alcohols (for example, methanol, ethanol, 1-propanol, 2-propanol, 1-butanol and 2-butanol), glycols (for example, 1,3-butylene glycol and propylene glycol), glycerin, ketones (for example, acetone and methyl ethyl ketone), ethers (for example, diethyl ether, dioxane, tetrahydrofuran and propyl ether), acetonitrile, esters (for example, ethyl acetate and butyl acetate), aliphatic hydrocarbons (for example, hexane, heptane and liquid paraffin), aromatic hydrocarbons (for example, toluene and xylene), halogenated hydrocarbons (for example, chloroform), or mixed solvents of two or more of these solvents.

A lemon extract is commercially available from Koei Kogyo Co., Ltd. (trade name: Hormofruit Lemon), and the product may be used. Also, a squeezed liquid obtained by squeezing lemon contains the active ingredient similar to that contained in a lemon extract, and therefore the juice of the lemon may be used instead of the lemon extract.

### Hydrolyzed Pea Protein

The hydrolyzed pea protein is a polypeptide obtained by hydrolyzing pea proteins, and contains a high polarity of acidic amino acids (aspartic acid and glutamic acid) and basic amino acids (arginine, histidine and lysine) in a large amount. Pea (scientific name: Pisum sativum L.) is an annual or biennial plant belonging to the family Leguminosae which is widely cultivated for edible use, and various hydrolyzed solutions can be obtained by hydrolyzing the pea. Promois WJ (trade name) is a hydrolyzed solution of pea proteins, which has a molecular weight of about 500 and is commercially available from Seiwa Kasei Co., Ltd., and can be used as a hydrolyzed pea protein.

There is no particular limitation on the mixing ratio of the olive leaf extract, lemon extract and hydrolyzed pea protein used in the present invention, and the mixing ratio can be optionally set. In order to make the carbonylation inhibiting ability of the protein carbonylation inhibitor according to the present invention more excellent, the mixing ratio of the extracts is preferably 0.00001% by mass or more and 10% by mass or less, more preferably 0.001% by mass or more and 5% by mass or less, and still more preferably 0.01% by mass or more and 1% by mass or less, based on the entire protein carbonylation inhibitor.

The following examples are presented in order to merely illustrate the embodiments of the invention, and should not be considered to limit the scope of the invention.

### EXAMPLES

### 1. Materials and Methods

### Subjects and Skin Samples

Normal aged facial skin samples were obtained from the perilesional, uninvolved portion of a surgical margin of excised skin tumors in 7 Japanese patients (3 males and 4 females, aged from 52 to 81 years (Table 1). Written informed consent was obtained from them before surgery. Such normal-appearing perilesional skin was evaluated with colorimetry before surgery, and it was isolated immediately after surgical resection of the tumors. The part was subjected to histochemical studies, while the remaining portion, after extensive washing with PBS to remove blood, was separated into the epidermis and dermis by incubation it in PBS containing 2M sodium bromide at 37° C. for 90 min. After further washing with PBS, the isolated epidermal sheet and the dermis were subjected to clinical observation and colorimetry.

Moreover, a various ages of skin samples from 13 Japanese, were obtained. Face samples (5 males and 5 females, 10-90 years old, Table 2) as sun-exposed areas, and back and buttock (2 males and 1 female, 15-69 years old, Table 2) as sun-protected areas were obtained. Immediately after surgical resection of their tumors, the normal-appearing peripheral skin was isolated and subjected to histochemical examinations as mentioned above.

All of these studies were conducted according to Helsinki Principles and were approved by the ethical committees of National Defense Medical College, the University of Electro-Communication, and Shiseido Research Center.

### Histochemistry

The presence of actinic elastosis was demonstrated with Elastica van Gieson stains or Weigert's resorcin-fuchsin stain. The degree of actinic elastosis was scored as described by Kligman [29], i.e., G0, no change; G1, increase in number without thickening; G2, greater hyperplasia with thickening and curling; G3, marked hyperplasia with thickening and curling, and frequent branching; and G4, complete replacement of the dermis by a dense tangle of thickened, disorderly fibers accompanied by disorganization into murky amorphous masses.

Carbonyl protein was detected by the reaction with 0.1 μM fluorescein-5-thiosemicarbazide (Molecular Probes, Eugene, Oreg., USA) in 0.1 M 2-morpholinoethane sulfonic acid-Na buffer (pH 5.5) for 4 h, being followed by observation under a fluorescence microscope (Olympus, Tokyo, Japan).

Acrolein adduct was immunohistochemically detected by using a monoclonal anti-acrolein antibody (5F6, Nippon Oil and Fat, Tokyo) as a primary antibody, and was followed by serial reactions with anti-mouse Ig conjugated to peroxidase polymer (DAKO, CA, USA) and DAB staining.

### Colorimetry

Skin color was measured by using a chromameter Konica-Minolta CM 2600d. The measurement of excised skin samples was performed by placing the skin samples on a white board. Data were expressed in the CIE L*a*b* color space, and the parameter b* was employed as a value for yellowish color.

### Measurement of Optical Properties of the Skin Samples

For precise measurements of the optical properties, excised skin samples were embedded into OCT compound (Sakura, Tokyo, Japan) and stored at −80° C. until being processed. Horizontally cut thin sections of the skin samples were prepared at approximately 100 μm thickness by using a cryomicrotome (Carl zeiss, Jena, Germany), followed by extensive washing by changing PBS several times to remove the OCT compound. These sections were processed to a round form with a diameter of 10 mm, and placed in a quartz cell having a well with a diameter and a depth of 10 mm and 100 μm, respectively. The thicknesses of these skin specimens were measured with an optical procedure using an optical coherence tomography (OCT) system, (ISIS optonics, Mannheim, Germany). From the OCT cross-section imaging, 5 lines of perpendicular to the interface of the specimens were drawn and an average length of vertically made 5 lines of the interface of the skin specimens was regarded as the thickness.

Optical diffuse transmittance and reflectance spectra of the specimens were measured by using a UV-3150 spectrophotometer (SHIMADZU Corp., Kyoto, Japan) equipped with an integrating sphere. The scattering phase functions were measured with a TPM-2500 multi-spectro gonio photometer (TECH WORLD, Saitama, Japan). The spectra of the relative scattering and absorption coefficients of the specimens were obtained by an inverse Monte Carlo method from the measured spectra of the optical diffuse transmittances and reflectances with the measured scattering phase functions and the thicknesses of the specimens [29b, 29c].

### Acellular Dermis Model

A dermal equivalent model consisted of contracted collagen gel containing a trace amount of other extracellular matrix was prepared according to the method described by Tsunenaga et al. [30] with a slight modification. Briefly, dermal fibroblasts (1×10⁶ cells) were cultivated in 10 ml of Dulbecco's modified Eagle's medium supplemented with 10% fetal bovine serum and 0.3% acid-solubilized bovine type I collagen (Koken, Tokyo, Japan) in a petri dish of 60 mm diameter at 37° C. and 5% CO₂. After 7 days of culture, a contracted collagen gel approximately 15 mm diameter was produced. Thereafter, this dermal equivalent model was extensively washed with serial changes of an excess amount of de-ionized water to destroy fibroblasts and to remove soluble materials.

### Induction of Carbonyl Modification

The prepared acellular dermis model was incubated in 2.5 ml of an aqueous solution of chemical reagents, containing acrolein (50 μM to 10 mM) and 4-hydroxynonenal (4-HNE, 50 μM to 10 mM) for the carbonyl modification, and ribose (200 mM) and glyoxal (10 mM) for glycation. They were INCUBATED at 37° C. and the solutions were changed twice a week. Changes taking place in their color were monitored by colorimetry.

### 2. Results

### Yellowish Change of the Dermis Observed in the Sun-Exposed Area

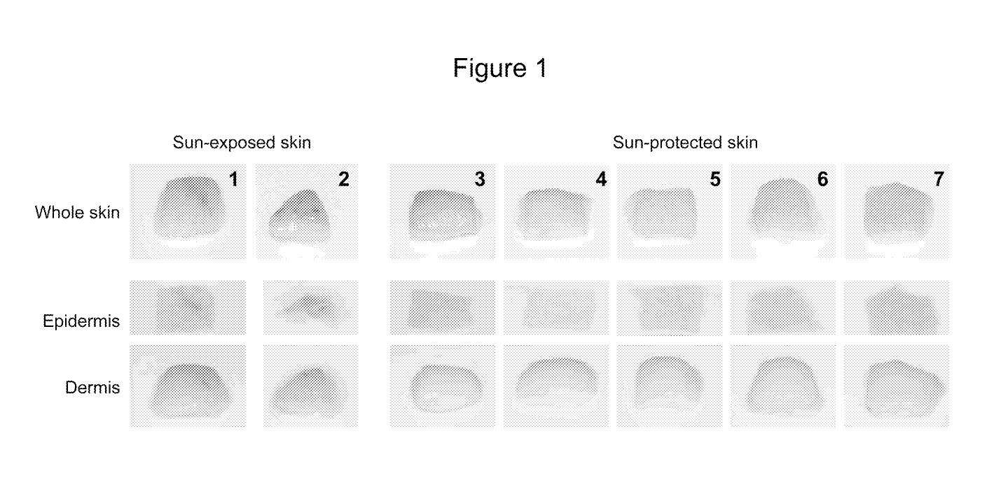

When the yellow skin color parameter b* was measured in vivo, as well as ex vivo after excision, higher b* values were consistently noted in the samples from the sun-exposed facial skin (as compared with those obtained from the covered skin of the groin or buttock) (Table 1). To exclude the influence of the epidermal component, such as melanin to this color characteristics, the inventors further measured the color of the isolated dermis, which clearly demonstrated higher b* values in the dermis of the sun-exposed skin than that of the dermis obtained from the sun-protected skin areas (Table 1). FIG. 1 definitely shows, the yellowish appearance observed in the dermis obtained from the sun-exposed skin.

TABLE 1
Skin yellowness of sun-exposed and sun-protected skin.

| | | | Yellowness evaluated by b*² | | |
|---|---|---|---|---|---|
| | | | | Ex vivo | |
| | | | In vivo | full-thickness | Separated |
| Case no¹ | Age/sex | Site | skin | skin | dermis |
| 1 | 73/F | Face | 24.2 | 24.7 | 27.7 |
| 2 | 68/M | Face | 21.6 | 23.3 | 25.4 |
| 3 | 59/F | Chest | n.d. | 23.4 | 14.2 |
| 4 | 81/F | Groin | 15.3 | 16.3 | 15.0 |
| 5 | 70/F | Groin | 19.5 | 18.2 | 15.0 |
| 6 | 52/M | Buttock | 19.2 | 17.2 | 15.5 |
| 7 | 56/M | Buttock | 14.6 | 18.6 | 19.7 |

¹Cases 1 and 2 indicate sun-exposed skin and cases 3-7 indicate sun-protected skin.
²Blue-yellow chromaticity coordicnate in the commission internationale de l'Eclairage (CIE)-L*a*b* color space.
F, female;
M, male;
n.d., not determined.

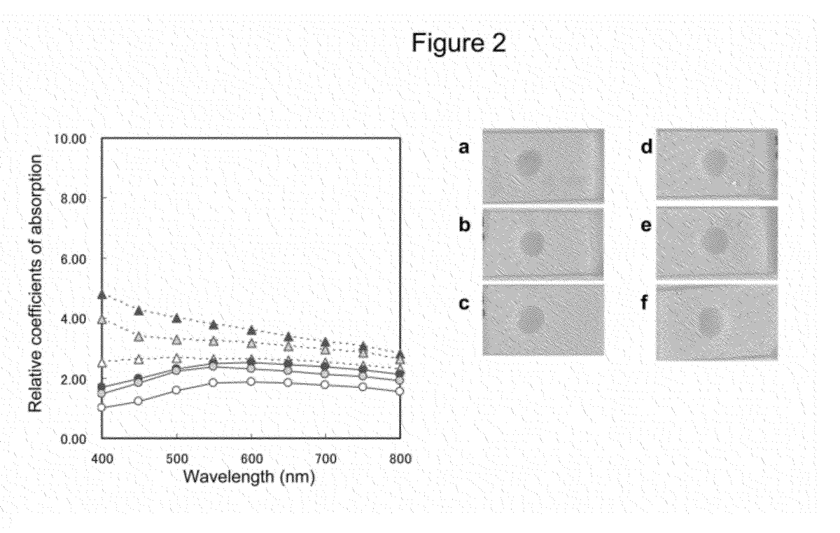

To examine the depth dependency of the yellowish color of the dermis, the inventors prepared a horizontal section of approximately 100 μm in thickness from those excised skin, and measured their optical properties. As shown in FIG. 2, the horizontal sections obtained from the sun-exposed areas showed much higher values in the short-wavelength region than those obtained from the sun-protected areas, indicating that the short-wavelength region was related to the yellowish color change. Most of all, the sections obtained from the upper-most layer of the sun-exposed dermis showed the highest values, indicating that the yellowish color alteration was most prominent in the most superficial portion of the dermis of the sun-exposed skin.

### Increased Carbonyl Modification in the Sun-Exposed Dermis

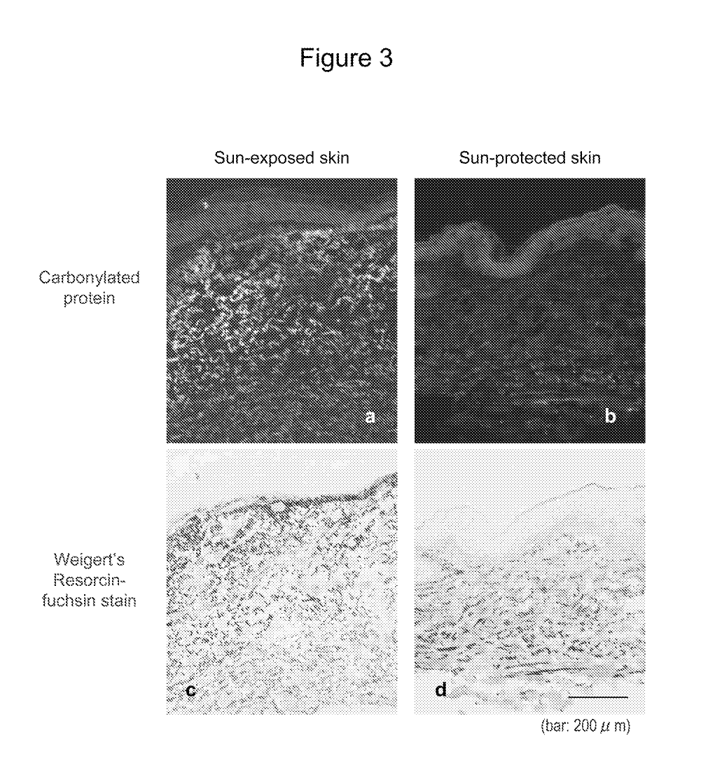

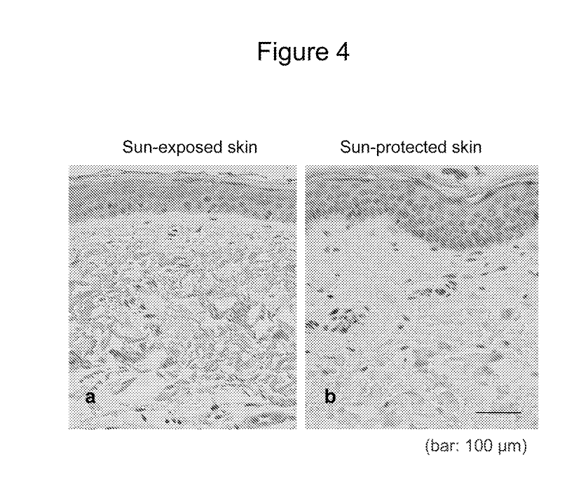

The inventors carried out histochemical examination with fluorescein-labeled hydrazide to detect carbonyl modification in the skin. These specimens clearly showed that the uppermost layer of the dermis of the sun-exposed skin exhibited apparently the highest carbonyl modification with a slight decrease with the depth of the dermis. One of such typical findings was shown in FIG. 3. In contrast, the dermis obtained from the sun-protected areas showed little carbonyl modification. Moreover, the localization of carbonyl modification was found to almost correspond to that of elastic fibers, as detected by simultaneously performed carbonyl protein staining and Weigert's resorcin-fuchsin staining, suggesting that the protein components of elastic fiber could be one of those susceptible to carbonyl modification in the sun-exposed dermis. Immunohistochemical examination with anti-acrolein antibody revealed that a significant positive staining was observed in the extracellular matrix of the sun-exposed dermis, but not in the sun-protected dermis (FIG. 4). These results suggested that acrolein is one of the aldehydes introduced into sun-exposed dermal protein.

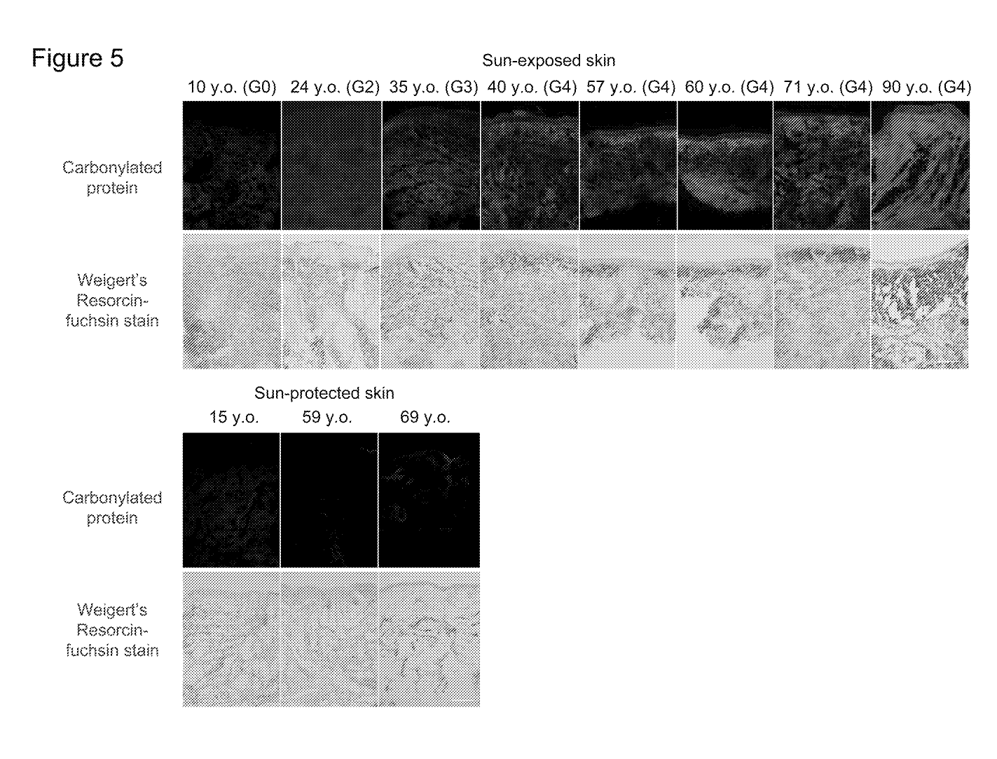

To clarify the relationship between the accumulation of carbonyl modification and the degree of actinic elastosis, a serial skin sections were prepared from the facial skin of various aged subjects. As shown in FIG. 5 and Table 2, apparent accumulation of carbonyl protein was detected in the skin exhibiting actinic elastosis of grade 4, while no such significant carbonyl protein was detected in the sun-protected skin even in those obtained from aged individuals (FIG. 5 and Table 3). These findings strongly suggest that the carbonyl modification in the dermis is closely associated with chronic photodamage.

TABLE 2
Degree of actinic elastosis and accumulation
of carbonyl protein in sun-exposed skin.

| | | Degree of | |
|---|---|---|---|
| | | actinic | Carbonyl |
| Age/sex | Site | elastosis¹ | stain |
| 10/M | Face | G0 | − |
| 20/F | Face | G1 | − |
| 24/M | Face | G2 | − |
| 35/F | Face | G3 | − |
| 40/M | Face | G4 | + |
| 57/M | Face | G4 | + |
| 60/F | Face | G4 | + |
| 70/F | Face | G4 | + |
| 71/M | Face | G4 | + |
| 90/F | Face | G4 | + |

¹Degree of actinic elastosis (G0-G4) was scored according to the criteria by Kligman [29].

TABLE 3
Negative detection of carbonyl protein in sun-protected skin

| | | Carbonyl |
|---|---|---|
| Age/sex | Site | stain |
| 15/M | Back | − |
| 59/M | Buttock | − |
| 69/F | Buttock | − |

### Experimental Induction of the Yellowish Change in a Dermal Model by Carbonyl Modification

To confirm a link suspected between the carbonyl modification and the yellowish change of the dermis, the inventors examined the effect of experimentally produced carbonyl modification on the color of an acellular dermis model, and selected a contracted collagen gel as a dermis model, and treated it with acrolein or 4-HNE to induce carbonyl modification, or with d-ribose or glyoxal to induce glycation as a reference. The treatment with 10 mM acrolein or 10 mM 4-HNE resulted in the production of a significant yellowish change in the gel, while only a slight yellowish change was produced with the treatment with 200 mM d-ribose or 10 mM glyoxal (FIGS. 6 A and B). These results suggested that the carbonyl modification can efficiently accelerate yellowish colorization in the dermal protein rather than glycation. Furthermore, a long term treatment of the dermis model with 50 μM acrolein or 50 μM 4-HNE, which were almost equivalent to those of the physiological concentrations in human tissue, also led to the yellowish colorization (FIGS. 6 C and D). It is of interest that the yellowish change of the dermal model treated with 50 μM acrolein showed an almost linear increase depending on the treatment period, while the yellowish change induced with 50 μM 4-HNE reached the maximum in the first 10 days.

### Evaluation of Carbonylation Inhibitory Effect of Extracts

For the test, 96-well plates previously coated with type I collagen were used (Corning Inc.; Code number: NCO 3585). Acrolein (AccuStandard Inc.) which is an aldehyde derived from a lipid peroxide was used as a reagent for inducing carbonylation. When the acrolein acts on a protein, a carbonyl group is introduced. The carbonyl group introduced was detected using biotin-hydrazide (Pierce Inc.; Code number: 21339) which binds to the carbonyl group and is obtained by linking a hydrazine group (—NHNH₂) and biotin. With respect to the carbonylation inhibitory effect of each extract (lemon juice extract, olive leaf extract and hydrolyzed pea protein), to what extent the introduction of the carbonyl group was inhibited was investigated by comparing detection efficiency of the carbonyl group in the presence and absence of the extract.

Specifically, 300 μM acrolein and each extract were dissolved to prepare a sample solution. The concentration of each extract was set at 0.01% to 0.3%. In a sample solution not containing the extract, 300 μM acrolein and a solvent in an amount equal to that of the extract were dissolved (positive control). Each 100 μL of these sample solutions were dispensed into each well of the collagen plates and reacted at 37° C. all day and night. Also, a similar test was carried out using a solution containing no acrolein and containing a solvent in an amount equal to that of the extract (negative control).

The plates reacted all day and night were washed with a phosphate buffer solution containing a 0.1% Tween 20 surfactant (PBS-T buffer). Then, each 100 μL of a 0.1 μM biotin-hydrazide solution prepared in a 100 mM 2-morpholinoethanesulfonic acid monohydrate (MES) buffer solution was dispensed into each well and reacted at room temperature for 2 hours. After the reaction, the well was washed with the PBS-T buffer. Then, each 100 μL of a 0.1 μg/mL horseradish peroxidase (HRP)-labeled avidin (Vector Inc.; A-2004) was dispensed into each well and reacted at 37° C. for 1 hour. After the well was washed with the PBS-T buffer solution, color development was carried out using a substrate solution (Bio-Rad Inc.; product number: 172-1066) containing 3,3′,5,5′-tetramethylbenzidine hydrochloride (TMB) which is a substrate of HRP. The color reaction was stopped with a 1M sulfuric acid solution. Then, absorbance at a wavelength of 450 nm was measured to quantify the amount of proteins into which a carbonyl group was introduced.

The carbonylation inhibitory effect was expressed by (absorbance of sample−absorbance of negative control)/(absorbance of positive control−absorbance of negative control). Evaluation was carried out by n=5 for each concentration of each sample. Average values and standard deviation values were calculated from the resulting values, a t-test was carried out for two independent groups, and P values were obtained to evaluate significant differences. The statistically significant difference was expressed as follows: “*” has a significant difference of p<0.05 and “**” has a significant difference of p<0.01.

TABLE 4
Carbonylation Inhibitory Effect of Agents

| | | (% of control) |
|---|---|---|
| Agent name | Concentration | Average ± standard deviation |
| Lemon juice extract | 0.01% | 83.7 ± 12.3* |
| | 0.03% | 45.1 ± 27.2** |
| Olive leaf extract | 0.1% | 86.2 ± 8.2* |
| | 0.3% | 66.6 ± 6.6** |
| Pea extract | 0.1% | 100.8 ± 44.4 |
| | 0.3% | 62.7 ± 11.0** |

*Significant difference (p < 0.05) exists.
**Significant difference (p < 0.01) exists.

As is apparent from the above results, each extract (lemon juice extract, olive leaf extract and hydrolyzed pea protein) significantly inhibits carbonylation of proteins. Here, Hormofruit Lemon (trade name), which is commercially available from Koei Kogyo Co., Ltd., was used for evaluation of the lemon juice extract. An olive leaf extract, which is commercially available from Maruzen Pharmaceuticals Co., Ltd., was used for evaluation of the olive leaf extract. Promois WJ (trade name), which is commercially available from Seiwa Kasei Co., Ltd., was used for evaluation of the hydrolyzed pea protein.

### Evaluation of Action of Inhibiting Yellowing of Skin by Olive Leaf Extract

As an example of the evaluation, inhibition of yellowing by an olive leaf extract (Maruzen Pharmaceuticals Co., Ltd.) was evaluated. Olive (Olea europaea Linne) is an evergreen tree which belongs to the genus Olea of the family Magnoliaceae, and is now cultivated over the world in a region at a relatively high ambient temperature and around Shodo island in Japan. An olive oil obtained by squeezing ripe fruits is widely used for edible, medicinal and cosmetic applications, and the olive leaf is known to have antiseptic, antipyretic and antihypertensive actions and recently used as a material for tea. However, it was not hitherto known that the olive leaf extract has a carbonylation inhibiting action. Since the olive leaf extract was colored by itself, pigment components were removed using active charcoal so as to not prevent color evaluation, and the resulting extract was used to carry out experiments. Specifically, the dermis model prepared as described above was immersed in 2.5 mL of a solution (control) containing only 1 mM acrolein and a solution containing 1 mM acrolein and 10% of the olive leaf extract, which were prepared using a phosphate buffer solution. Then, the treated dermis model was incubated at 37° C. for 6 days. The solutions were exchanged for those readjusted every day. The color change of the dermis model was measured using Konica-Minolta Chroma Meter CM-2600d, diffuse lighting, 8° direction light-receiving φ3 mm. When measuring the color change, the dermis model was removed from the solutions and mounted on a white tile for correction (CR-A43 (1849-701)). The parameter b* (yellowness) in the CIE L*a*b* color space calculated from the resulting spectral reflectance spectrum was used as an indicator of the yellowing.

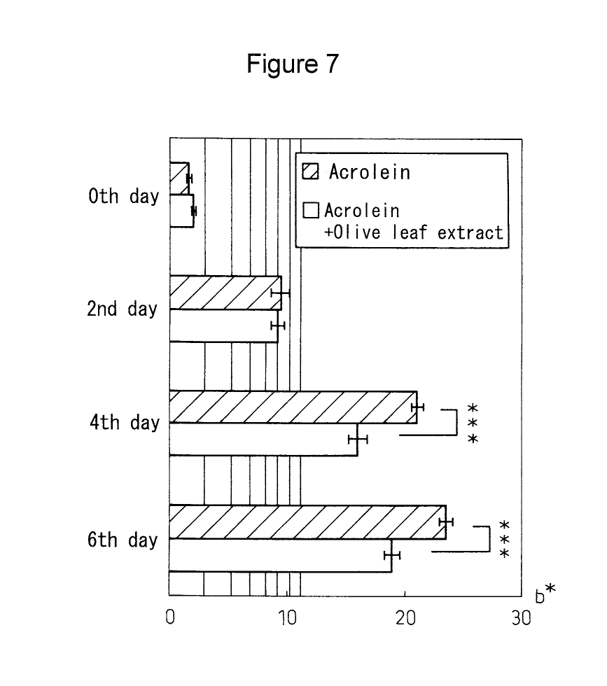

As is apparent from FIG. 7, yellowing of the dermis model was inhibited and significant inhibition of increase of the parameter b* was seen. This shows that the olive leaf extract has a very high possibility to have an effect of inhibiting the yellowing of the skin.

### REFERENCES

1. Bleehen S S, Disorder of skin color. In “Textbook of Dermatology 6^{th} ed. (Champion R H, Burton J L, Burns D A, Breathnach S M eds.) pp. 1753, Blackwell Science Ltd. 1998.
2. Anderson R R, Parrish J A. The optics of human skin. J Invest Dermatol 1981; 77: 13-19
3. Takahashi M, Watanabe H, Kumagai H, Nakayama Y Physiological and morphological changes in facial skin with aging (II). A study on racial diffedrence. (In Japanese)
J Soc Cosmet Chem Japan 23(1); 22-30, 1989
4. Ochiai N, Yano K, Denda S, Akutsu N, Yanai M, Shimatani Y, Takasu E, Kono Y
Changes in skin physiological parameters in pre-, peri-, post-menopause and effect of hormone replacement therapy. (In Japanese)
Jpn Menopause Soc 8(1) 33-40, 2000
5. Nishimori Y, Pearse A D, Edwards C, Marks R Elastotic degenerative change and yellowish discolouration in photoaged skin.
Skin Res Technol 1998; 4: 79-82
6. Mizutari K, Ono T, Ikeda K, Kayashima K, Horiuchi S. Photo-enhanced modification of human skin elastin in actinic elastosis by N(epsilon)-(carboxymethyl)lysine, one of the glycoxidation products of the Maillard reaction.
   J Invest Dermatol. 1997; 108(5):797-802.
6b. Jeanmaire C, Danoux L, Pauly G.
Glycation during human dermal intrinsic and actinic ageing: an in vivo and in vitro model study
Br J Dermatol 2001; 145: 10-18
7. Tanaka N, Tajima S, Ishibashi A, Uchida K, Shigematsu T.
Immunohistochemical detection of lipid peroxidation products, protein-bound acrolein and 4-hydroxynonenal protein adducts, in actinic elastosis of photodamaged skin.
Arch Dermatol Res. 2001; 293(7):363-7.
8. Sander C S, Chang H, Salzmann S, Müller C S, Ekanayake-Mudiyanselage S, Elsner P, Thiele J J.
Photoaging is associated with protein oxidation in human skin in vivo.
J Invest Dermatol. 2002; 118(4):618-25.
9. Ulrich P, Cerami A.
Protein glycation, diabetes, and aging.
Recent Prog Horm Res. 2001; 56:1-21.
10. Meerwaldt R, Graaff R, Oomen P H, Links T P, Jager J J, Alderson N L, Thorpe S R, Baynes J W, Gans R O, Smit A J. Simple non-invasive assessment of advanced glycation endproduct accumulation.
Diabetologia. 2004; 47(7):1324-30.
11. Monnier V M, Sell D R, Genuth S.
Glycation products as markers and predictors of the progression of diabetic complications.
Ann N Y Acad. Sci. 2005; 1043:567-81.
12. Stevens J F, Maier C S.
Acrolein: sources, metabolism, and biomolecular interactions relevant to human health and disease.
Mol Nutr Food Res. 2008; 52(1):7-25.
13. Uchida K, Kanematsu M, Morimitsu Y, Osawa T, Noguchi N, Niki E.
Acrolein is a product of lipid peroxidation reaction. Formation of free acrolein and its conjugate with lysine residues in oxidized low density lipoproteins.
J Biol. Chem. 1998; 273(26):16058-66.
14. Uchida K, Kanematsu M, Sakai K, Matsuda T, Hattori N, Mizuno Y, Suzuki D, Miyata T, Noguchi N, Niki E, Osawa T. Protein-bound acrolein: potential markers for oxidative stress.
Proc Natl Acad Sci USA. 1998; 95(9):4882-7.
15. Petersen D R, Doorn J A.
Reactions of 4-hydroxynonenal with proteins and cellular targets. (Review)
Free Radic Biol Med. 2004; 37(7):937-45.
16. Siegel S J, Bieschke J, Powers E T, Kelly J W.
The oxidative stress metabolite 4-hydroxynonenal promotes Alzheimer protofibril formation.
Biochemistry. 2007; 46(6):1503-10.
17. Thiele J J, Schroeter C, Hsieh S N, Podda M, Packer L. The antioxidant network of the stratum corneum. Curr Probl Dermatol 2001; 29: 26-42.
18. Oliver C N, Ahn B W, Moerman E J, Goldstein S, Stadtman E R. Age-related changes in oxidized proteins. J Biol Chem 1987; 262: 5488-5491.
19. Stadtman E R. Protein oxidation and aging. Science 1992; 257: 1220-1224.
20. Berlett B S, Stadtman E R. Protein oxidation in aging, disease, and oxidative stress. J Biol Chem 1997; 272: 20313-20316.
21. Chevion M, Berenshtein E, Stadtman E R. Human studies related to protein oxidation: protein carbonyl content as a marker of damage. Free Radical Res 2000; 33: S99-108.
22. Grune T, Davies K J A. Oxidative processes in aging.
In: Masoro E J, Austad S N, eds. Handbook of the biology of aging, 5th edn. New York: Academic Press, 2001: 25-79.
23. Thiele J J, Hsieh S N, Briviba K, Sies H. Protein oxidation in human stratum corneum: susceptibility of keratins to oxidation in vitro and presence of a keratin oxidation gradient in vivo. J Invest Dermatol 1999; 113: 335-339.
24. Thiele J J, Traber M G, Re R, Espuno N, Yan L J, Cross C E, Packer L. Macromolecular carbonyls in human stratum corneum: a biomarker for environmental oxidant exposure? FEBS Lett 1998; 422: 403-406.
25. Fujita H, Hirao T, Takahashi M. A simple and non-invasive visualization for assessment of carbonylated protein in the stratum corneum. Skin Res Tech 2007; 13, 84-90.
26. Iwai I, Hirao T. Protein Carbonyls Damage the Water-Holding Capacity of the Stratum Corneum. Skin Pharmacol Physiol 2008; 21: 269-273.
27. Iwai I, Ikuta K, Murayama K, Hirao T. Change in optical properties of stratum corneum induced by protein carbonylation in vitro. Int J Cosmet Sci 2008; 30: 41-46.
28. Kobayashi Y, Iwai I, Akutsu N, Hirao T. Increased carbonyl protein levels in the stratum corneum of the face during winter. Int J Cosmet Sci 2008; 30: 35-40.
29. Kligman A M
Early destructive effect of sunlight on human skin.
J Am Med Assoc 210(13), 2377-2380, 1969
29b. Tuchin V, Utz S, Yaroslaysky I
Tissue optics, light distribution, and spectroscopy Optical Engineering 1994; 33(10): 3178-3188.
29c. Firbank M, Hiraoka M, Essenpreis M, Delpy D T, Measurement of the optical properties of the skull in the wavelength range 650-950 nm, Phys. Med. Biol. 1993; 38(4), 503-510.
30. Tsunenaga M, Adachi E, Amano S, Burgeson R E, Nishiyama T.
Laminin 5 can promote assembly of the lamina densa in the skin equivalent model.
Matrix Biol. 1998; 17(8-9):603-13.
31. Ritz-Timme S, Laumeier I, Collins M J.
Aspartic acid racemization: evidence for marked longevity of elastin in human skin.
Br J. Dermatol. 2003; 149(5):951-9.
32. Stone P J, LuceyE C, Snider G L, Franzblau C.
Distribution of elastin in hamsters and the turnover rates of different elastin pools.
Proc Soc Exp Biol Med. 1997; 215(1):94-101.
33. Verzijl N, DeGroot J, Thorpe S R, Bank R A, Shaw J N, Lyons T J, Bijlsma J W, Lafeber F P, Baynes J W, TeKoppele J M.
Effect of collagen turnover on the accumulation of advanced glycation end products.
J Biol Chem. 2000; 275(50):39027-31.
34. Márová I, Záhejský J, Sehnalová H.
Non-enzymatic glycation of epidermal proteins of the stratum corneum in diabetic patients.
Acta Diabetol. 1995; 32(1):38-43.
35. Ohshima H, Oyobikawa M, Tada A, Maeda T, Takiwaki H, Itoh M, Kanto H.
Melanin and facial skin fluorescence as markers of yellowish discoloration with aging.
Skin Res Technol. 2009; 15(4):496-502.

We claim:

1. A method for preventing or treating skin discoloration of a subject by inhibiting carbonylation of dermal proteins of the subject who is in need of prevention or treatment for skin discoloration.

2. The method according to claim 1, wherein the skin discoloration is yellowish skin discoloration.

3. The method according to claim 1, wherein the dermal proteins are present in the upper dermis.

4. The method according to claim 1, wherein the skin is sun-exposed skin.

5. The method according to claim 1, wherein the skin is facial skin.

6. The method according to claim 1, wherein the carbonylation is caused by aging, disease or oxidative stress.

7. The method according to claim 6, wherein the disease is actinic elastosis.

8. The method according to claim 6, wherein the oxidative stress is due to exposure to ultraviolet radiation, internal and/or external application of oxidative chemicals or chemical attack by reactive carbonyls derived from the degradation of lipid peroxides.

9. The method according to claim 1, wherein the dermal proteins are extracellular matrix proteins.

10. The method according to claim 9, wherein the extracellular matrix proteins are collagen and/or elastin.
